(19) United States
(12) Patent Application Publication
(10) Pub. No.: US 2023/0036150 A1
(43) Pub. Date: Feb. 2, 2023

(54) Tracking System

(71) Applicant: Northern Digital Inc., Waterloo (CA)

(72) Inventors: Andrew Donald Wiles, Waterloo (CA); Utsav Pardasani, Waterloo (CA); Jose Luis Alarcon Herrera, Waterloo (CA)

(21) Appl. No.: 17/877,117

(22) Filed: Jul. 29, 2022

Related U.S. Application Data
(60) Provisional application No. 63/227,799, filed on Jul. 30, 2021.

(51) Int. Cl. A61B 34/20

(52) U.S. Cl. CPC A61B 34/20; A61B 2034/2061; A61B 2034/2055; A61B 2090/397

(57) ABSTRACT

A system comprising: two or more tracking sensors that are configured to provide pose information, wherein the two or more tracking sensors include: a first tracking sensor configured to provide a reference coordinate system; and a second tracking sensor that resides in the reference coordinate system relative to the first tracking sensor; one or more segments of optical fiber affixed to the second tracking sensor, wherein the one or more segments of optical fiber are tracked relative to the second tracking sensor; an interrogator that is configured to read measurements from the one or more segments of optical fiber; and a computing device configured to execute an algorithmic method on data from the one or more segments of optical fiber, wherein measurements taken from the one or more segments of optical fiber are placed in the context of pose information from the two or more tracking sensors.

### CLAIM OF PRIORITY

This application claims priority under 35 USC § 119(e) to U.S. Patent Application Ser. No. 63/227,799, filed on Jul. 30, 2021, the entire contents of which are hereby incorporated by reference.

### TECHNICAL FIELD

This disclosure relates to a tracking system, and more particularly, to an electromagnetic tracking system.

### BACKGROUND

Electromagnetic Tracking (EMT) systems are used to aid in locating instruments and anatomy in medical procedures. These systems utilize a magnetic transmitter in proximity to one or more magnetic sensors. The one or more sensors can be spatially located relative to the transmitter.

### SUMMARY

Tracking systems that provide pose (i.e., position and orientation) information in medical procedures are used to locate instruments and make measurements with respect to patient anatomy. Tracking systems may comprise of several tracking sensors which provide position and/or orientation (i.e., pose) information. The medical procedures supported by tracking systems span many domains and can include: surgical interventions, diagnostic procedures, imaging procedures, and radiation treatment. The most common offerings are either a) based on tools that are uniquely identifiable and tracked by optical cameras or b) electromagnetic sensors that measure a field from a magnetic field generator.

In an aspect, a system includes two or more tracking sensors that are configured to provide pose information, wherein the two or more tracking sensors include a first tracking sensor configured to provide a reference coordinate system, and a second tracking sensor that resides in the reference coordinate system relative to the first tracking sensor. The system includes one or more segments of optical fiber affixed to the second tracking sensor, wherein the one or more segments of optical fiber are tracked relative to the second tracking sensor, an interrogator that is configured to read measurements from the one or more segments of optical fiber, and a computing device configured to execute an algorithmic method on data from the one or more segments of optical fiber, wherein measurements taken from the one or more segments of optical fiber are placed in the context of pose information from the two or more tracking sensors.

Implementations can include one or more of the following features.

In some implementations, the two or more tracking sensors share the reference coordinate system.

In some implementations, the two or more tracking sensors share a global coordinate system.

In some implementations, the system is an electromagnetic tracking system.

In some implementations, the system is an optical tracking system.

In some implementations, multiple tracking sensors are situated along the one or more segments of optical fiber.

In some implementations, pose information of the multiple tracking sensors are combined with measurements from the one or more segments of optical fiber in a sensor fusion algorithm to enhance the accuracy of the pose measurements of the tracking sensors.

In some implementations, the sensor fusion algorithm is based on Kalman filtering, extended Kalman filtering, invariant Kalman filtering, particle filtering, or weighted averaging.

In some implementations, the sensor generates pose information for its sensors from camera images.

In some implementations, the system calculates the pose of its sensors by an algorithm that processes camera images of the sensors.

In some implementations, the system also includes markers that are retroreflective and reflect infrared light emitted by the system.

In some implementations, the tracking system generates pose information for its sensors based on magnetic measurements.

In some implementations, the tracking system generates pose information for its sensors based on ultrasonic measurements.

In some implementations, the one or more segments of optical fiber are instrumented with one or more Fiber Bragg Gratings to produce strain measurements.

In some implementations, a strain of the one or more segments of optical fiber is determined by principles of Rayleigh scattering.

In some implementations, measurements of the one or more segments of optical fiber are compensated for by temperature measurements.

In some implementations, the two or more tracking sensors instrument one or more guidewires.

In some implementations, data from the one or more segments of optical fiber is processed by an algorithmic method to provide shape measurements along its length and registered in the same co-ordinate space as the first tracking sensor and the second tracking sensor.

In some implementations, data from the one or more segments of optical fiber is processed by an algorithmic method to provide pose information along the length of the one or more segments of optical fiber and registered in the same co-ordinate space as the first tracking sensor and the second tracking sensor.

In some implementations, an algorithmic method on the computing device actively synchronizes data capture from the system with the data acquisition of the one or more segments of optical fiber.

In some implementations, the one or more segments of optical fiber are about 20-30 cm in length.

In some implementations, the system also includes a field generator.

In some implementations, the field generator resides on the back of a patient.

In some implementations, a shape of the one or more segments of optical fiber is tracked by the system.

In some implementations, the location of the second tracking sensor is determined relative to the reference coordinate system.

In some implementations, the system also includes a control unit and a sensor interface configured to control the field generator and determine the pose of the first tracking sensor and the second tracking sensor.

In some implementations, the system is configured for use in a surgical environment.

In some implementations, one or more sensors are affixed to one or more of a deep brain stimulation electrode, a biopsy needle, a catheter, a guidewire, a sheath, and a transeptal needle.

In some implementations, one or more sensors are affixed to an interventional neurovascular device such as a stent retriever, reperfusion catheter, coil placement devices, coil assist devices, flow diverters, balloons, and delivery devices for embolic materials.

In some implementations, one or more sensors are affixed to an implant deployment device, an endoscope, or a bronchoscope.

In another aspect, a computing device implemented method includes receiving pose information from a first electromagnetic sensor affixed to a patient, the pose information defining a reference coordinate system, receiving pose information from a second electromagnetic sensor affixed to a guidewire instrumented by an optical fiber, receiving shape information from the optical fiber, determining a pose of the second electromagnetic sensor relative to the reference coordinate system, wherein the pose of the second electromagnetic sensor is determined from the pose information received from the second electromagnetic sensor, and determining a shape of the optical fiber from the shape information received from the optical fiber, wherein the shape of the optical fiber is aligned (e.g., registered) with the pose of the second electromagnetic sensor.

Implementations can include one or more of the following features.

In some implementations, the guidewire instrumented by an optical fiber includes the optical fiber affixed to the guidewire.

In some implementations, the guidewire instrumented by an optical fiber includes the optical fiber encapsulated by the guidewire.

In some implementations, the computing device implemented method includes providing a shape of the guidewire.

The details of one or more embodiments of the subject matter described herein are set forth in the accompanying drawings and the description below. Other features, objects, and advantages of the subject matter will be apparent from the description and drawings, and from the claims.

### DESCRIPTION OF DRAWINGS

FIGS. 1A and 1B are schematic diagrams of an example Electromagnetic Tracking (EMT) system.

FIG. 2 is a flowchart showing an exemplary method that could be performed by the EMT system of FIGS. 1A and 1B.

FIG. 3 shows a schematic diagram of an example computer system.

Like reference numbers and designations in the various drawings indicate like elements.

### DETAILED DESCRIPTION

Tracking Systems (e.g., measurement systems that provide pose information) can be used in medical applications (e.g., tracking medical equipment in surgical theaters) to track one or more objects (e.g., a medical device, one or more robotic arms, etc.), thereby determining and identifying the respective three-dimensional location, orientation, pose, etc. of the object or objects for medical professionals (e.g., a surgeon). In some implementations, tracking can be in the form of visualizations or representations of the pose information. In some implementations, such tracking provides guidance to professionals, e.g., in image-guided procedures, and in some cases may reduce reliance on other imaging modalities, such as fluoroscopy, which can expose the patient to ionizing radiation that can create health risks.

In some implementations, the tracking system is an electromagnetic tracking system, an optical tracking system, etc. and can employ both electromagnetic and optical components.

In general, in tracking systems that include electromagnetic tracking functionality, a transmitter having one or more coils is configured to generate an EM field (e.g., an alternating current (AC) field). A sensor having one or more coils that is in proximity to the generated EM field is configured to measure characteristics of the generated EM field. The measured characteristics of the EM field dependent upon on the position and orientation of the sensor relative to the transmitter. For example, when the sensor is located at a particular position and orientation, the EM field at that particular location may have particular characteristics. The sensor can measure the characteristics of the EM field and provide measurement information to a computing device such as a computer system (e.g., one or more signals produced by the sensor can provide the measurement information). Using the measurement information present on the received sensor signals, the computing device can determine the position and/or orientation of the sensor. By employing this technique, the position, orientation, etc. of a medical device (e.g., containing the sensor, attached to the sensor, etc.) can be determined and processed by the computing device (e.g., the computing device identifies the position and location of a medical device and graphically represents the medical device, the sensor, etc. in images such as registered medical images, etc.).

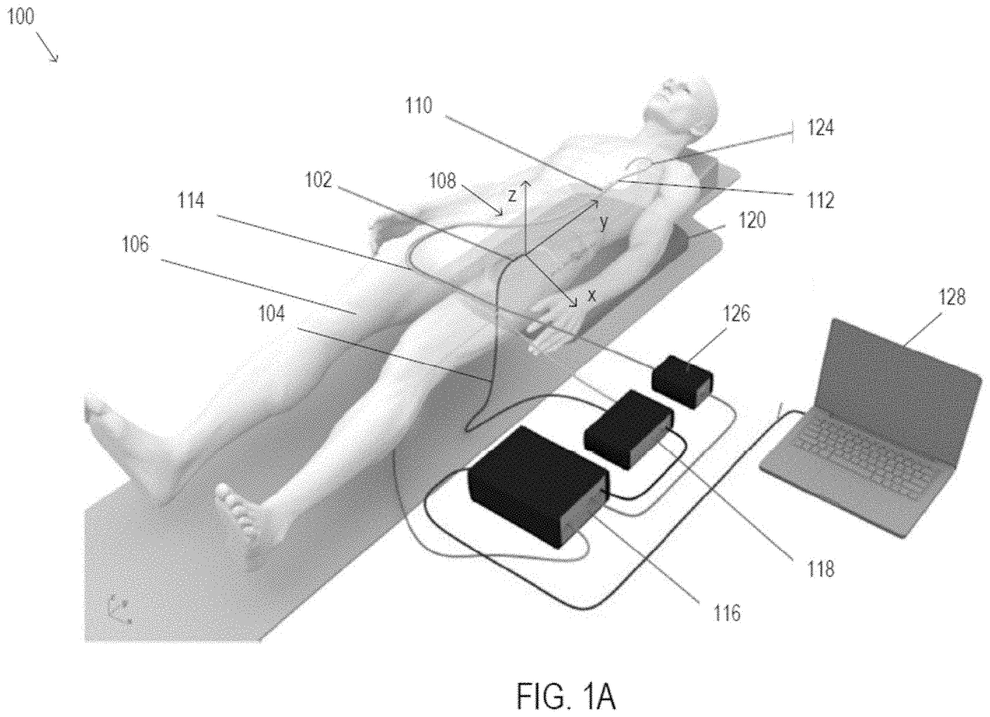

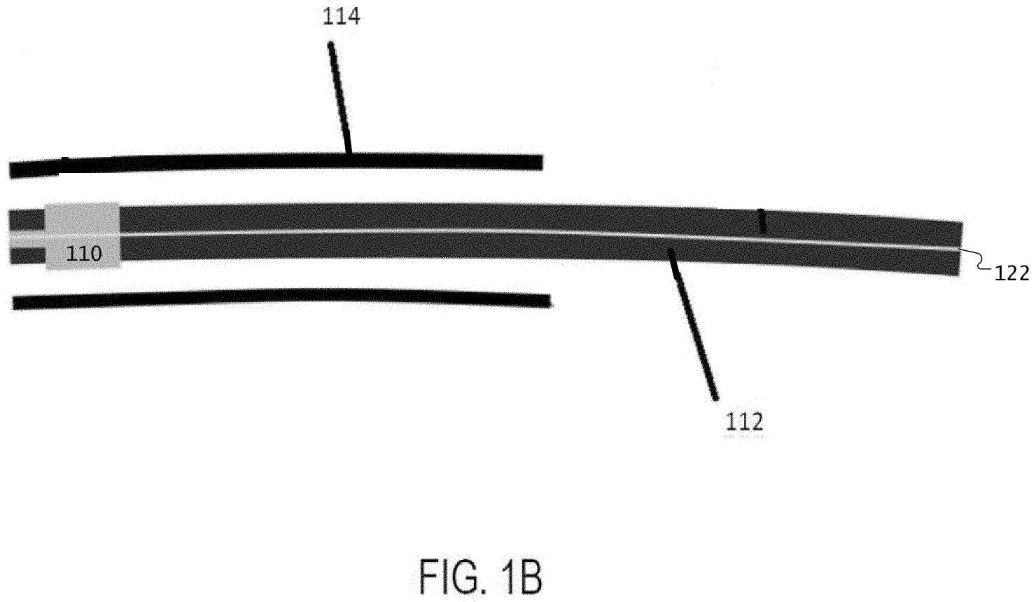

FIGS. 1A and 1B show an example of an electromagnetic tracking (EMT) system 100 that is implemented in the surgical environment (e.g., a surgical theater). The system 100 is configured to determine the location of one or more electromagnetic sensors associated with medical devices (e.g., scalpels, probes, etc.), equipment, etc. For example, one or more sensors can be embedded in a guidewire for tracking the guidewire in various medical procedures involving a patient. The electromagnetic tracking techniques employed for tracking guidewires, for example, may be similar to those described in U.S. patent application Ser. No. 13/683,703, entitled “Tracking a Guidewire”, filed on Nov. 21, 2012, which is hereby incorporated by reference in its entirety.

In this example, an electromagnetic tracking system 100 includes an electromagnetic sensor 102 (e.g., a 6DOF sensor) or multiple sensors embedded in a leading segment of a wire 104 that is attached to a patient 106. In some arrangements, a sensor (e.g., a 6DOF sensor) or sensors can be positioned in one or more different positions along the length of the wire 104. For example, multiple sensors can be distributed along the length of the wire 104.

The electromagnetic sensor 102 can be attached to the patient and used to define a reference coordinate system 108. Pose information (e.g., coordinates, poses, etc.) about further sensors can be determined relative to this reference coordinate system, which is defined relative to the patient.

In some implementations (e.g., implementations that include multiple sensors), by establishing the reference coordinate system 108 and using electromagnetic tracking, a location and orientation of another tracking sensor 110 (e.g., a second 6DOF sensor) or multiple sensors embedded in a leading segment of a guidewire 112 can be determined relative to the reference coordinate system 108. That is, the pose of the sensor 110 is realized relative to the patient 106 because the reference coordinate system 108 is defined relative to the patient. In some implementations, a catheter 114 is inserted over the guidewire after the guidewire is inserted into the patient.

In this particular implementation, a control unit 116 and a sensor interface unit 118 are configured to resolve system signals to determine the pose of the sensors 102, 110 using electromagnetic tracking methodologies. For example, the sensor 102 can be connected to the sensor interface unit 118, e.g., through a wire 104. Similar to electromagnetic systems, optical based systems may employ techniques for tracking and identifying the respective three-dimensional location, orientation, etc. of objects for medical professionals. Or, in the case where the tracking system 100 employs optical tracking capabilities, the pose of the sensors 102, 110 can be determined using optical tracking methodologies.

A field generator 120 resides beneath the patient (e.g., located under a surface that the patient is positioned on, embedded in a table that the patient lays upon—such as a table top, etc.) to emit electromagnetic fields that are sensed by the accompanying electromagnetic sensors 102, 110. In some implementations, the field generator 120 is an NDI Aurora Tabletop Field Generator (TTFG), although other field generator techniques and/or designs can be employed.

The pose (i.e., the position and/or orientation) of a tracked sensor (e.g., the first tracking sensor 102, the second tracking sensor 110) refers to a direction the tracked object is facing with respect to a global reference point (e.g., the reference coordinate system 108), and can be expressed similarly by using a coordinate system and represented, for example, as a vector of orientation coordinates (e.g., azimuth (ψ), altitude (θ), and roll (φ) angles). The tracking system 100 operates to determine a shape of an optical fiber, as discussed below. Additionally, the tracking system 100 operates to be an up to six degree of freedom (6DOF) measurement system that is configured to allow for measurement of position and orientation information of a tracked sensor related to a forward/back position, up/down position, left/right position, azimuth, altitude, and roll. For example, if the second tracking sensor 110 includes a single receiving coil, a minimum of at least five transmitter assemblies can provide five degrees of freedom (e.g., without roll). In an example, if the second tracking sensor 110 includes at least two receiving coils, a minimum of at least six transmitter assemblies can provide enough data for all six degrees of freedom to be determined. Additional transmitter assemblies or receiving coils can be added to increase tracking accuracy or allow for larger tracking volumes.

The guidewire 112 is instrumented by (e.g., affixed to, encapsulating, etc.) an optical fiber 122. The optical fiber 122 can form one or multiple shapes as it extends into and through the body of the patient. Light that is transmitted down the fiber can be used to track the location, orientation, (i.e., pose) of segments of the fiber, and as an outcome fiber shape information can be determined. In the example shown in FIG. 1A, a hook shape 124 is produced by the optical fiber 122, which can be resolved by the system 100. A starting location of a segment of the fiber 122 is known by the system 100 due to the pose of the second electromagnetic sensor 110 being known. The fiber 122 can be tracked relative to the second electromagnetic sensor 110 based on a fiber optic signal. For example, an interrogator 126 will receive fiber optic signals (e.g., a waveform that is reflected back) reflected through the optical fiber 122. The received fiber optic signals can be used to determine the mechanical strain along the length of the fiber, and therefore also its shape. For example, the shape of the segment of fiber 122 may be determined based on the fiber optic signals transmitted to and from the interrogator 126.

In some implementations, fiber optic tracking may be limited to local tracking. A reference coordinate system is provided by the first electromagnetic sensor 102. The location and orientation of the second electromagnetic sensor 110 is known due to electromagnetic tracking. Thus, only the segment of fiber 122 that extends beyond the second electromagnetic sensor 110 must be tracked. For example, the control unit 116 and sensor interface unit 118 can resolve system signals to determine the pose of the sensors 102, 110 using electromagnetic tracking methodologies, discussed further below. Shape information from optical fiber 122 can then be fused with the pose information of electromagnetic sensors 110 and 102 on computing device 128 and can be computed in the patient reference frame (e.g., in the reference coordinate system 108). In doing so, the shape information can be further processed for visualization with other data that is registered to the patient reference, for example manually created annotations or medical images collected prior to or during the procedure.

In some implementations, the interrogator 126 is an optoelectronic data acquisition system that provides measurements of the light reflected through the optical fiber. The interrogator provides these measurements to the computing device (e.g., the computing device 128).

At each periodic refraction change due to the shape of the optical fiber, a small amount of light is reflected. All the reflected light signals combine coherently to one large reflection at a particular wavelength when the grating period is approximately half the input light's wavelength. This is referred to as the Bragg condition, and the wavelength at which this reflection occurs is called the Bragg wavelength. Light signals at wavelengths other than the Bragg wavelength, which are not phase matched, are essentially transparent. In general, a fiber Bragg grating (FBG) is a type of distributed Bragg reflector constructed in a relative short segment of optical fiber that reflects particular wavelengths of light and transmits the light of other wavelengths. An FBG can be produced by creating a periodic variation in the refractive index of a fiber core, which produces a wavelength-specific dielectric mirror. By employing this technique, a FBG can be used as an inline optical fiber for sensing applications.

Therefore, light propagates through the grating with negligible attenuation or signal variation. Only those wavelengths that satisfy the Bragg condition are affected and strongly back-reflected. The ability to accurately preset and maintain the grating wavelength is a fundamental feature and advantage of Fiber Bragg Gratings.

The central wavelength of the reflected component satisfies the Bragg relation: λ_{Bragg}=2 nΛ, with n the index of refraction and A the period of the index of refraction variation of the FBG. Due to the temperature and strain dependence of the parameters n and A, the wavelength of the reflected component will also change as function of temperature and/or strain. This dependency can be utilized for determining the temperature or strain from the reflected FBG wavelength.

In some implementations, the sensor 102 provides a reference coordinate system 108 for system 100 that may be affixed to the patient 106. The reference coordinate system is defined by a suitable location on the patient 106. The location, orientation, etc. of the guidewire 112 can be defined within the reference coordinate system. In this way, the shape of the fiber can be tracked relative to the patient anatomy. In some implementations, the guidewire 112 may include NDI Aurora magnetic sensors or be tracked by NDI's optical tracking systems.

In some cardiac applications the shape of the optical fiber 122 can be used to support medical procedures. For example, the shape of the segment of optical fiber 122 can provide information about a transeptal puncture operation in the context of a mitral valve repair/replacement or a catheter across an atrial septum wall for atrial fibrillation treatment. Additionally, the shape of the fiber optics line 122 can be used to cannulate the vessel entering the kidney from the aorta for a stent placement.

Tracking systems are frequently accompanied by computing equipment, such as the computing device 128, which can process and present the measurement data. For example, in a surgical intervention, a surgical tool measured by the tracking system can be visualized with respect to the anatomy marked up with annotations from the pre-operative plan. Another such example may include an X-ray image annotated with live updates from a tracked guidewire.

Medical procedures that are supported by tracking systems frequently make measurements with respect to a reference co-ordinate system located on the patient. In doing so, medical professionals can visualize and make measurements with respect to the patient anatomy and correct for gross patient movement or motion. In practice, this is accomplished by affixing an additional tracking sensor (e.g., a 6DOF sensor) to the patient. This is also accomplished by sensing the shape of the fiber 122.

The described tracking systems can be advantageous because they do not require line-of-sight to the objects that are being tracked. That is, they do not require a directly unobstructed line between tracked tools and the camera for light to pass. In some implementations, the described systems have improved metal immunity and immunity to electrical interference. That is, they do not require minimal presence of metals and sources of electrical noise in their vicinity to provide consistent tracking performance.

In medical procedure contexts where the approach of a surgical or endoscopic tool can improve patient outcomes, additional intraoperative imaging modalities can be used such as Ultrasound, MRI, or X-rays. Another advantage of the described tracking systems (e.g., the system 100 of FIG. 1) is that the shape of the surgical/endoscopic tool is not distorted by imaging artifacts. Also, EMT systems do not require direct manual control of an imaging probe by a skilled practitioner to maintain the quality of visualization. Further, the present systems do not expose the patient and medical staff to ionizing radiation. Thus the number of workflow contexts that stand to benefit from this technology is vast, covering a variety of endovascular procedures, electrophysiology, structural heart interventions, peripheral vascular interventions, bronchoscopic interventions, endoscopic procedures, neurosurgical interventions, biopsy needle guidance, percutaneous coronary interventions, transcatheter embolization procedures, pain management procedures, urological interventions, robotic laparoscopic interventions, and others.

There are techniques by which optical transducers built into an optical fiber can produce measurements (for example wavelength) that can be used to estimate pose information along the length of the fiber. In some implementations, the optical fiber may be equipped with a series of Fiber Bragg Gratings (FBG), which amount to a periodic change in the refractive index manufactured into the optical fiber. In some implementations, the optical fiber may simply rely on Rayleigh scattering, which is a natural process arising from microscopic imperfections in the fiber. Techniques using FBG, Rayleigh scattering, or both have the capacity to reflect specific wavelengths of light that may correspond to strain or changes in temperature within the fiber. Deformations in the fiber cause these wavelengths to shift, and the wavelength shift can be measured by a system component known as the interrogator. The technology used by the interrogator 126 to measure wavelength shift can be Wavelength-Division Multiplexing (WDM) or Optical Frequency-Domain Reflectometry (OFDR). In doing so, the shape of the fiber can be estimated by an algorithm running on a computing device. By affixing a fiber instrumented as such, a new sensing/measurement paradigm is possible for 6DOF tracking systems, enabling the pose and shape measurements along the fiber in the co-ordinate space of the 6DOF tracking system. Additionally, in an OT supported procedure, this can allow one to take pose measurements outside of the measurement volume or line-of-sight of the OT system. In the context of an EMT supported procedure, this can allow one to take pose measurements in a region with high metal distortion where EMT sensors would normally perform poorly, or one can use the fiber measurements to correct for electromagnetic/metal distortion.

While FIGS. 1A and 1B are largely directed to a system 100 that includes electromagnetic components (e.g., an electromagnetic tracking system), it should be understood that the one or more tracking sensors (e.g., 6DOF sensors) described herein may be part of other (e.g., different) systems, such as optical tracking systems that include one or more cameras and one or more tracking sensors.

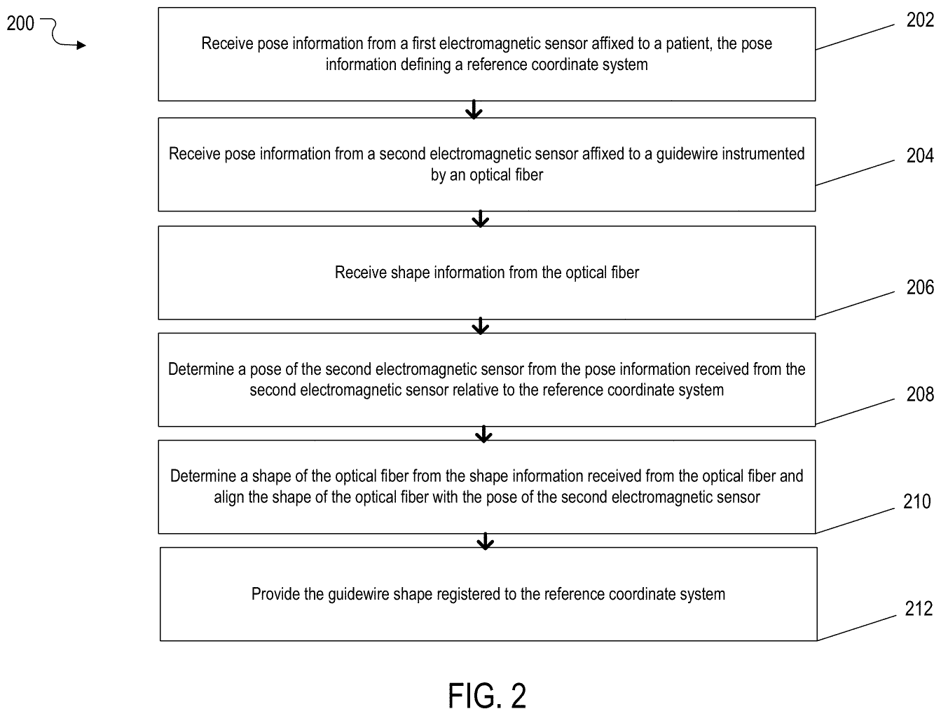

FIG. 2 shows a flowchart of an exemplary method 200 that could be performed by the system 100 (e.g., the computing system 128 of the system 100) of FIGS. 1A and 1B.

At 202, pose information is received from a first electromagnetic sensor affixed to a patient. For example, the first electromagnetic sensor 102 can be affixed to the patient 106, as described above. The pose information defines a reference coordinate system (e.g., the reference coordinate system 108). For example, the pose information of the first electromagnetic sensor may comprise position information (e.g., expressed as three dimensional cartesian co-ordinates, etc.), and/or orientation information (e.g., expressed as quaternions, Euler rotation angles, rotation matrices, etc.).

At 204, pose information is received from a second electromagnetic sensor affixed to a guidewire instrumented by (e.g., affixed to, encapsulating, etc.) an optical fiber. For example, the pose information of the first electromagnetic sensor may comprise position information (e.g., expressed as three dimensional cartesian co-ordinates, etc.), and/or orientation information (e.g., expressed as quaternions, Euler rotation angles, rotation matrices, etc.). As described above, the guidewire 112 includes the second electromagnetic sensor 110 and an optical fiber (e.g., the optical fiber 122). The optical fiber may run along the guidewire. A segment of the optical fiber (e.g., the optical fiber 122) extends past the second electromagnetic sensor.

At 206, shape information is received from the optical fiber. For example, an interrogator (e.g., the interrogator 126) can receive fiber optic signals (e.g., a waveform that is reflected back) reflected through the optical fiber 122. The received fiber optic signals can be used to determine shape information along the length of the fiber.

At 208, the pose of the second electromagnetic sensor is determined from the pose information received from the second electromagnetic sensor relative to the reference coordinate system.

At 210, the shape of the optical fiber is determined from the shape information received from the optical fiber. For example, fiber optic signals can be used to determine the mechanical strain along the length of the fiber, and therefore also its shape, as discussed above. Also, the shape of the optical fiber is aligned with (e.g., registered to) the pose of the second electromagnetic sensor. For example, the segment of fiber that extends past the second electromagnetic sensor is tracked and aligned with the second electromagnetic sensor in the reference coordinate system. In some implementations, the entire optical fiber is tracked and aligned with second electromagnetic sensor in the reference coordinate system. In some examples, a fiber optic interrogator (e.g., the interrogator 126 of FIG. 1A) can be used to resolve the shape of the segment of fiber.

At 212, a shape of the guidewire is registered in the reference co-ordinate system affixed to the patient. For example, shape information from the optical fiber can be fused with the pose information of the electromagnetic sensors and presented in a patient reference frame (e.g., in the reference coordinate system 108). The shape of the guidewire can be provided, e.g., as a visualization with respect to the anatomy marked up with annotations from the pre-operative plan. Another such example may include an X-ray image annotated with live visualizations from the tracked guidewire shape and pose information. Consequently, in computing the fibre shape in the reference-coordinate system, meaningful visualizations of the guidewire can be created from other data that is registered to the reference coordinate system such as medical image data or annotations created before the procedure.

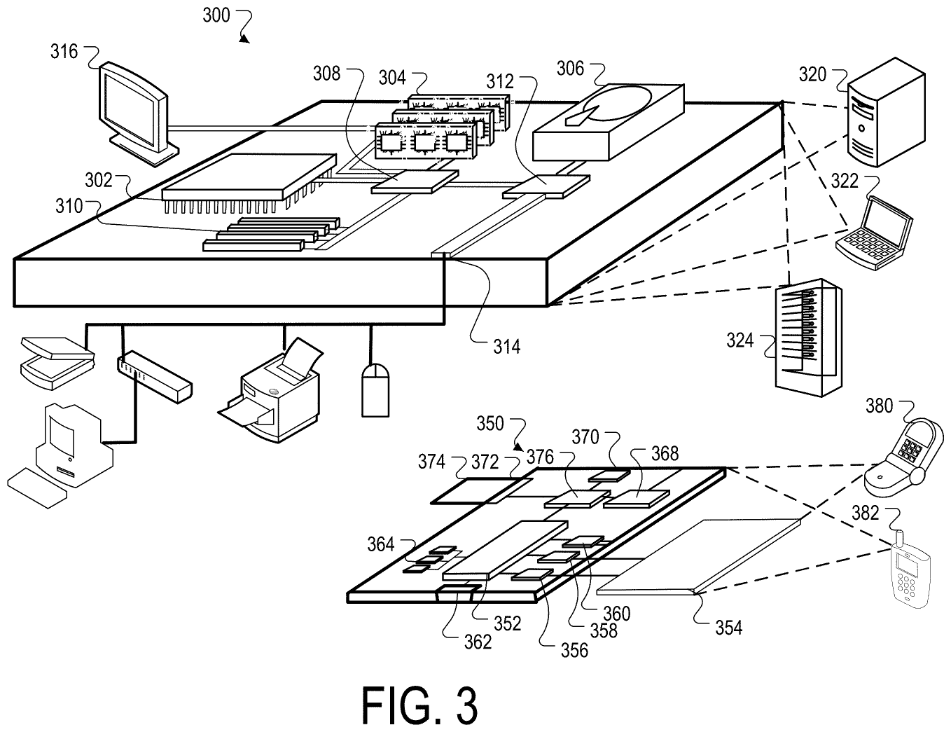

As described above, the operation of the system 100 can be controlled by a computing device 128. In particular, the computing device 128 can be used to interface with the system 100 and cause the locations/orientations of the electromagnetic sensors 102, 110 and the optical fiber 122 to be determined. FIG. 3 shows an example computing device 300 and an example mobile computing device 350, which can be used to implement the techniques described herein, such as the method 200 of FIG. 2. For example, the computing device 300 may be implemented as the computing device 128 of FIG. 1A. Computing device 300 is intended to represent various forms of digital computers, including, e.g., laptops, desktops, workstations, personal digital assistants, servers, blade servers, mainframes, and other appropriate computers. Computing device 350 is intended to represent various forms of mobile devices, including, e.g., personal digital assistants, cellular telephones, smartphones, and other similar computing devices. The components shown here, their connections and relationships, and their functions, are meant to be examples only, and are not meant to limit implementations of the techniques described and/or claimed in this document.

Computing device 300 includes processor 302, memory 304, storage device 306, high-speed interface 308 connecting to memory 304 and high-speed expansion ports 310, and low speed interface 312 connecting to low speed bus 314 and storage device 306. Each of components 302, 304, 306, 308, 310, and 312, are interconnected using various busses, and can be mounted on a common motherboard or in other manners as appropriate. Processor 302 can process instructions for execution within computing device 300, including instructions stored in memory 304 or on storage device 306, to display graphical data for a GUI on an external input/output device, including, e.g., display 316 coupled to high-speed interface 308. In some implementations, multiple processors and/or multiple buses can be used, as appropriate, along with multiple memories and types of memory. In addition, multiple computing devices 300 can be connected, with each device providing portions of the necessary operations (e.g., as a server bank, a group of blade servers, a multi-processor system, etc.).

Memory 304 stores data within computing device 300. In some implementations, memory 304 is a volatile memory unit or units. In some implementation, memory 304 is a non-volatile memory unit or units. Memory 304 also can be another form of computer-readable medium, including, e.g., a magnetic or optical disk.

Storage device 306 is capable of providing mass storage for computing device 300. In some implementations, storage device 306 can be or contain a computer-readable medium, including, e.g., a floppy disk device, a hard disk device, an optical disk device, a tape device, a flash memory or other similar solid state memory device, or an array of devices, including devices in a storage area network or other configurations. A computer program product can be tangibly embodied in a data carrier. The computer program product also can contain instructions that, when executed, perform one or more methods, including, e.g., those described above. The data carrier is a computer- or machine-readable medium, including, e.g., memory 304, storage device 306, memory on processor 302, and the like.

High-speed controller 308 manages bandwidth-intensive operations for computing device 300, while low speed controller 312 manages lower bandwidth-intensive operations. Such allocation of functions is an example only. In some implementations, high-speed controller 308 is coupled to memory 304, display 316 (e.g., through a graphics processor or accelerator), and to high-speed expansion ports 310, which can accept various expansion cards (not shown). In some implementations, the low-speed controller 312 is coupled to storage device 306 and low-speed expansion port 314. The low-speed expansion port, which can include various communication ports (e.g., USB, Bluetooth®, Ethernet, wireless Ethernet), can be coupled to one or more input/output devices, including, e.g., a keyboard, a pointing device, a scanner, or a networking device including, e.g., a switch or router (e.g., through a network adapter).

Computing device 300 can be implemented in a number of different forms, as shown in FIG. 3. For example, the computing device 300 can be implemented as standard server 320, or multiple times in a group of such servers. The computing device 300 can also can be implemented as part of rack server system 324. In addition or as an alternative, the computing device 300 can be implemented in a personal computer (e.g., laptop computer 322). In some examples, components from computing device 300 can be combined with other components in a mobile device (e.g., the mobile computing device 350). Each of such devices can contain one or more of computing device 300, 350, and an entire system can be made up of multiple computing devices 300, 350 communicating with each other.

Computing device 350 includes processor 352, memory 364, and an input/output device including, e.g., display 354, communication interface 366, and transceiver 368, among other components. Device 350 also can be provided with a storage device, including, e.g., a microdrive or other device, to provide additional storage. Components 350, 352, 364, 354, 366, and 368, may each be interconnected using various buses, and several of the components can be mounted on a common motherboard or in other manners as appropriate.

Processor 352 can execute instructions within computing device 350, including instructions stored in memory 364. The processor 352 can be implemented as a chipset of chips that include separate and multiple analog and digital processors. The processor 352 can provide, for example, for the coordination of the other components of device 350, including, e.g., control of user interfaces, applications run by device 350, and wireless communication by device 350.

Processor 352 can communicate with a user through control interface 358 and display interface 356 coupled to display 354. Display 354 can be, for example, a TFT LCD (Thin-Film-Transistor Liquid Crystal Display) or an OLED (Organic Light Emitting Diode) display, or other appropriate display technology. Display interface 356 can comprise appropriate circuitry for driving display 354 to present graphical and other data to a user. Control interface 358 can receive commands from a user and convert them for submission to processor 352. In addition, external interface 362 can communicate with processor 342, so as to enable near area communication of device 350 with other devices. External interface 362 can provide, for example, for wired communication in some implementations, or for wireless communication in some implementations. Multiple interfaces also can be used.

Memory 364 stores data within computing device 350. Memory 364 can be implemented as one or more of a computer-readable medium or media, a volatile memory unit or units, or a non-volatile memory unit or units. Expansion memory 374 also can be provided and connected to device 350 through expansion interface 372, which can include, for example, a SIMM (Single In Line Memory Module) card interface. Such expansion memory 374 can provide extra storage space for device 350, and/or may store applications or other data for device 350. Specifically, expansion memory 374 can also include instructions to carry out or supplement the processes described above and can include secure data. Thus, for example, expansion memory 374 can be provided as a security module for device 350 and can be programmed with instructions that permit secure use of device 350. In addition, secure applications can be provided through the SIMM cards, along with additional data, including, e.g., placing identifying data on the SIMM card in a non-hackable manner.

The memory 364 can include, for example, flash memory and/or NVRAM memory, as discussed below. In some implementations, a computer program product is tangibly embodied in a data carrier. The computer program product contains instructions that, when executed, perform one or more methods. The data carrier is a computer- or machine-readable medium, including, e.g., memory 364, expansion memory 374, and/or memory on processor 352, which can be received, for example, over transceiver 368 or external interface 362.

Device 350 can communicate wirelessly through communication interface 366, which can include digital signal processing circuitry where necessary. Communication interface 366 can provide for communications under various modes or protocols, including, e.g., GSM voice calls, SMS, EMS, or MMS messaging, CDMA, TDMA, PDC, WCDMA, CDMA2000, or GPRS, among others. Such communication can occur, for example, through radio-frequency transceiver 368. In addition, short-range communication can occur, including, e.g., using a Bluetooth®, WiFi, or other such transceiver (not shown). In addition, GPS (Global Positioning System) receiver module 370 can provide additional navigation- and location-related wireless data to device 350, which can be used as appropriate by applications running on device 350.

Device 350 also can communicate audibly using audio codec 360, which can receive spoken data from a user and convert it to usable digital data. Audio codec 360 can likewise generate audible sound for a user, including, e.g., through a speaker, e.g., in a handset of device 350. Such sound can include sound from voice telephone calls, recorded sound (e.g., voice messages, music files, and the like) and also sound generated by applications operating on device 350.

Computing device 350 can be implemented in a number of different forms, as shown in FIG. 3. For example, the computing device 350 can be implemented as cellular telephone 380. The computing device 350 also can be implemented as part of smartphone 382, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include one or more computer programs that are executable and/or interpretable on a programmable system. This includes at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described herein can be implemented on a computer having a display device (e.g., a CRT (cathode ray tube) or LCD (liquid crystal display) monitor) for presenting data to the user, and a keyboard and a pointing device (e.g., a mouse or a trackball) by which the user can provide input to the computer. Other kinds of devices can be used to provide for interaction with a user as well. For example, feedback provided to the user can be a form of sensory feedback (e.g., visual feedback, auditory feedback, or tactile feedback). Input from the user can be received in a form, including acoustic, speech, or tactile input.

The systems and techniques described here can be implemented in a computing system that includes a backend component (e.g., as a data server), or that includes a middleware component (e.g., an application server), or that includes a frontend component (e.g., a client computer having a user interface or a Web browser through which a user can interact with an implementation of the systems and techniques described here), or a combination of such backend, middleware, or frontend components. The components of the system can be interconnected by a form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the components described herein can be separated, combined or incorporated into a single or combined component. The components depicted in the figures are not intended to limit the systems described herein to the software architectures shown in the figures.

A number of embodiments have been described. Nevertheless, it will be understood that various modifications may be made without departing from the spirit and scope of the disclosure. Accordingly, other embodiments are within the scope of the following claims.

What is claimed is:

1. A system comprising:
two or more tracking sensors that are configured to provide pose information, wherein the two or more tracking sensors include:
a first tracking sensor configured to provide a reference coordinate system; and
a second tracking sensor that resides in the reference coordinate system relative to the first tracking sensor;
one or more segments of optical fiber affixed to the second tracking sensor, wherein the one or more segments of optical fiber are tracked relative to the second tracking sensor;
an interrogator that is configured to read measurements from the one or more segments of optical fiber; and
a computing device configured to execute an algorithmic method on data from the one or more segments of optical fiber, wherein measurements taken from the one or more segments of optical fiber are placed in the context of pose information from the two or more tracking sensors.

2. The system of claim 1, wherein the two or more tracking sensors share the reference coordinate system.

3. The system of claim 1, wherein the system is an electromagnetic tracking system or an optical tracking system.

4. The system of claim 1, wherein multiple tracking sensors are situated along the one or more segments of optical fiber.

5. The system of claim 4, wherein pose information of the multiple tracking sensors are combined with measurements from the one or more segments of optical fiber in a sensor fusion algorithm to enhance the accuracy of the pose measurements of the tracking sensors.

6. The system of claim 1, wherein the sensor generates pose information for its sensors from camera images.

7. The system of claim 1, further comprising markers that are retroreflective and reflect infrared light emitted by the system.

8. The system of claim 1, wherein the tracking system generates pose information for its sensors based on electromagnetic field measurements.

9. The system of claim 1, wherein the one or more segments of optical fiber comprise one or more Fiber Bragg Gratings.

10. The system of claim 1, wherein Rayleigh scattering is used to determine a strain of the one or more segments of optical fiber.

11. The system of claim 1, wherein measurements of the one or more segments of optical fiber are compensated for by temperature measurements.

12. The system of claim 1, wherein data from the one or more segments of optical fiber is processed by the computing device to provide shape measurements along its length and registered in the same co-ordinate space as the first tracking sensor and the second tracking sensor.

13. The system of claim 1, wherein data from the one or more segments of optical fiber is processed by the computing device to provide pose information along the length of the one or more segments of optical fiber and registered in the same co-ordinate space as the first tracking sensor and the second tracking sensor.

14. The system of claim 1, wherein the location of the second tracking sensor is determined relative to the reference coordinate system.

15. The system of claim 1, further comprising a control unit and a sensor interface configured to control a field generator and determine the pose of the first tracking sensor and the second tracking sensor.

16. The system of claim 1, wherein the two or more tracking sensors are 6DOF sensors.

17. A computing device implemented method comprising:
receiving pose information from a first electromagnetic sensor affixed to a patient, the pose information defining a reference coordinate system;
receiving pose information from a second electromagnetic sensor affixed to a guidewire instrumented by an optical fiber;
receiving shape information from the optical fiber;
determining a pose of the second electromagnetic sensor relative to the reference coordinate system, wherein the pose of the second electromagnetic sensor is determined from the pose information received from the second electromagnetic sensor; and
determining a shape of the optical fiber from the shape information received from the optical fiber, wherein the shape of the optical fiber is aligned with the pose of the second electromagnetic sensor.

18. The computing device implemented method of claim 17, wherein the guidewire instrumented by the optical fiber comprises the optical fiber affixed to the guidewire.

19. The computing device implemented method of claim 17, wherein the guidewire instrumented by the optical fiber comprises the optical fiber encapsulated by the guidewire.

20. The computing device implemented method of claim 17, further comprising providing a shape of the guidewire.
